(19) United States
(12) Patent Application Publication
(10) Pub. No.: US 2025/0174308 A1
(43) Pub. Date: May 29, 2025

(54) METHOD FOR PREDICTING ADVERSE REACTIONS BETWEEN DRUGS BASED ON MULTI-ATTRIBUTE AND MULTI-KERNEL REPRESENTATION LEARNING

(71) Applicant: UNIVERSITY OF ELECTRONIC SCIENCE AND TECHNOLOGY OF CHINA, Chengdu (CN)

(72) Inventors: Yongguo LIU, Chengdu (CN); Jiajing ZHU, Chengdu (CN); Qiaoqin LI, Chengdu (CN); Yun ZHANG, Chengdu (CN); Zhi CHEN, Chengdu (CN)

(73) Assignee: UNIVERSITY OF ELECTRONIC SCIENCE AND TECHNOLOGY OF CHINA, Chengdu (CN)

(21) Appl. No.: 18/648,495

(22) Filed: Apr. 29, 2024

(30) Foreign Application Priority Data
Nov. 29, 2023 (CN) 2023116173052

(51) Int. Cl. G16C 20/10; G16C 20/70

(52) U.S. Cl. CPC G16C 20/10; G16C 20/70

(57) ABSTRACT

A method for predicting adverse reactions between drugs based on multi-attribute and multi-kernel representation learning is provided. Aiming at existing differences of different drug properties in revealing a potential characteristic of adverse reactions between drugs and preference and tendency of different kernel functions themselves in calculating attribute representation similarities between drugs, the present invention, based on multi-attribute representations of drugs, provides a multi-kernel representation learning model, designs a distance learning strategy of kernel functions and a reconstruction strategy of the kernel functions, selects a representative kernel function, constructs an optimal kernel function combination by incidence relation between the representative kernel functions and original kernel function, reveals a relationship between the multi-attribute similarities of the drugs and the adverse reactions between drugs, and realizes prediction of the adverse reactions between drugs based on multi-attribute and multi-kernel representations.

### CROSS REFERENCE TO THE RELATED APPLICATIONS

This application is based upon and claims priority to Chinese Patent Application No. 2023116173052, filed on Nov. 29, 2023, the entire contents of which are incorporated herein by reference.

### TECHNICAL FIELD

The present invention belongs to the technical field of prediction of adverse reactions between drugs, and particularly relates to a method for predicting adverse reactions between drugs based on multi-attribute and multi-kernel representation learning.

### BACKGROUND

Adverse reactions between drugs mean that the drug efficacy or the pharmacological action of one drug is destroyed by the other drug after two drugs are combined in use, which causes changes in original in-vivo metabolic and absorption processes of the drugs, generating adverse reactions or toxic and side effects harmful to human bodies. For example, the combined use of a triazole antifungal agent voriconazole, and a synthesized adrenal corticosteroid will induce adverse reactions such as osteoporosis, ichorrhemia, and attenuation of vision. The common use of a polyamine sevelamer and a sulfomethylaminobenzoic acid derivative furosemide will cause adverse reactions such as bradycardia, asystole, and adynamic ileus. Identification and prediction of the adverse reactions between drugs have received attention gradually by scientific researchers in various disciplines, which are also a research hotspot in the current field of clinical pharmacy and medical treatment.

To address the problem of the adverse reactions between drugs, pharmaceutical enterprises all over the world have invested much capital in a new drug research and development stage for clinical tests before the drug is listed, so that the risk of the adverse reactions of the new listed drug is reduced, and the safety of the drug is improved. Despite this, there are still adverse reaction events happening frequently every year. The costs of the pharmaceutical enterprises in a safety monitoring stage for the new listed drug are increased, and the risk for the drug being discarded and withdrawn is also improved. On the one hand, the factors affecting the adverse reactions between drugs are complicated and diverse. For example, there are differences between biochemical attributes (molecular structure, target, enzyme, pathway, and side effect) of different drugs and the absorptive and metabolic capabilities of patients to drugs are also different, resulting in that extensive analysis and researches cannot be carried out in clinical tests for the various using conditions of the drugs and different human body states. On the other hand, there are a large number of drugs, leading to their huge combination space when they are used together, leading to high clinical test cost and long time consumption in clinical tests. Therefore, efficient identification and prediction of the adverse reactions between drugs cannot be conducted on a large scale by the clinical test methods, and it is not enough to find potential adverse reactions between drugs merely dependent on the clinical tests before new drug is listing. Therefore, predicting the adverse reactions between drugs makes up for the limitation of the clinical test methods and hysteresis of safety monitoring after the new drug is listed, thereby lowering the research and development cost of the new drug and the safety monitoring cost of the new listed drug.

Research work for predicting the adverse reactions between drugs at present is mainly divided into two categories: methods for predicting adverse reactions between drugs based on a knowledge base and methods for predicting adverse reactions between drugs based on drug attributes.

Public medical and sanitary institutions collect drug safety monitoring data submitted by drug users and medical workers through a spontaneous reporting system to construct a knowledge base for the adverse reactions between drugs. Researchers proposed the methods for predicting adverse reactions between drugs based on a knowledge base to detect the adverse reactions between drugs in biochemical texts, electronic medical records, and a biological heterogeneous database.

With deep research of pharmaceutical experts on drug attributes (molecular structure, side effect, target, enzyme, pathway, phenotype, gene, diseases, and the like), the researchers extract drug attribute information from the heterogeneous database of drugs, design the methods for predicting adverse reactions between drugs based on drug attributes, and reveal attribute rules of the adverse reactions of the drugs. Common research methods include multi-task learning, feature selection, graph convolutional neural networks, et al.

Although the above methods lay a good foundation for predicting the adverse reactions between drugs, they have the following defects: by calculating the similarities of multi-attribute representations between drugs by similarity measure functions, the current methods model the relationship of adverse reactions between drugs. Specifically, the similarity measure function can be regarded as a kernel function that first maps the attribute representations of the drugs to a high-dimensional space by using a projection strategy and then calculates inner products of the attributes representations of the two drugs in high-dimensional space representations, which is seen as adverse reaction scores between the drugs. Due to that different attributes are usually distinct in revealing the underlying characteristics of adverse reactions between drugs, the kernel function most compatible with its potential characteristics is preferably selected to model the adverse reactions between drugs. The current methods usually make attempts at various kernel functions (such as linear kernel, Gaussian kernel, and Sigmoid kernel), and select the kernel function with the best result as the optimal kernel function to measure the attribute similarity measure between drugs. However, because the potential characteristics of different drug attributes are diverse and complex, the selected optimal kernel function is limited by its representation capability, and the optimal kernel function only can approximate the potential characteristics of the attributes and cannot fully reflect the characteristics of the attributes. On the other hand, from the perspective of the kernel function, different kernel functions have their own preference and tendencies when calculating the attribute representation similarities between drugs.

Due to that different attributes are usually distinct in revealing the underlying characteristics of adverse reactions between drugs, and different kernels typically have their respective preferences and tendencies in similarity estimation, based on multiple attributes of the drugs for predicting the adverse reactions between drugs, it is proposed to construct the optimal kernel function combination to better reveal the potential characteristics of different attributes in modeling the adverse reactions between drugs, that is, to find an integrated similarity measure function which is capable of being compatible to diverse and complex characteristics of the different attributes. Specifically, this optimal kernel function combination integrates preference and tendencies of various kernel functions in calculating the multi-attribute similarities, which can better reveal the relationship between the multi-attribute similarities of the drugs and the adverse reactions between drugs, thereby improving the accuracy of predicting the adverse reactions between drugs. For the differences of multi-attributes in revealing the potential characteristics of the adverse reactions between drugs, the problem to be solved in the present invention is to construct the optimal kernel function combination for accurate prediction of adverse reactions between drugs.

### SUMMARY

To address the above problems, the present invention provides a method for predicting adverse reactions between drugs based on multi-attribute and multi-kernel representation learning. Based on multi-attribute information (molecular structure, target, pathway, side effect, phenotype, and disease) of the drugs, in view of highly dimensional and sparse multi-attribute feature space of the drugs and divergence among feature dimensions of different attributes, the method in the present invention includes: first, projecting a multi-attribute feature space of the drugs to a same low-dimension and dense feature space to learn their shared and private representations; then developing a multi-kernel representation learning model, and designing a distance learning strategy and a reconstruction strategy of the kernel functions by calculating an incidence relationship among the kernel functions so as to select representative kernel functions; and finally, constructing an optimal kernel function combination based on the incidence relationship between representative kernel functions and the original kernel functions for revealing the relationship between the multi-attribute similarities of the drugs and the adverse reactions between drugs, so as to predict the adverse reactions between drugs.

A technical solution for the present invention is as follows:

A method for predicting adverse reactions between drugs based on multi-attribute and multi-kernel representation learning, including the following steps:
S1: collecting data of adverse reactions between drugs and multi-attribute information of the drugs to construct vectors of adverse reactions between drugs and multi-attribute information of the drugs, specifically including: defining a drug set as D={d₁, d₂, . . . , d_{N}}, where N is the number of drugs; constructing a vector r^{ij}∈{0, 1}^{K} to denote an adverse reaction relationship between the i^{th} drug dᵢ and the j^{th} drug dⱼ, where K denotes the number of the types of adverse reactions, and if the k^{th} adverse reaction is induced by the interaction between; dᵢ and dⱼ, then rₖ^{ij}=1; otherwise, rₖ^{ij}=0, k=1, 2, . . . , K; and constructing a matrix X^{m}∈R^{N×L}^{ₘ} to denote a feature space of m^{th} attribute of the drug, where Lₘ denotes a feature dimension of the m^{th} attribute, m=1, 2, . . . , M, and M denotes the number of attributes;
S2: learning the shared and private representations of the multi-attribute information of the drugs, where the shared representation means that different attributes have consistency information for prediction of adverse reactions between drugs, the private representation means that different attributes contain specific supplementary information of each attribute, the feature space of each attribute consists of the shared and private representations after the multi-attribute feature spaces of the drugs are projected to a same low-dimensional dense space, and an objective function is constructed based on multi-attribute representation learning so as to obtain solutions of the shared and private representations of M attribute spaces of the drugs;
S3: constructing a distance learning strategy of kernel functions and a reconstruction strategy of the kernel functions, specifically including: using the shared and private representations of the drugs as an input of a kernel function set, performing similarity measures on the shared and private representations between two drugs by the kernel function set to calculate the distances among different kernel functions, in which the smaller the distances among different kernel functions is, the greater the similarity is, and thus obtaining a similarity matrices of the shared and private representations by using the distances among the kernel functions of the shared and private representations of drugs, and finally, constructing a kernel function learning strategy according to the similarity matrices and a kernel function incidence matrix, where the kernel function incidence matrix is a probability matrix in which the entries of the matrix denote the probability of one kernel function to represent another kernel function; and the reconstruction strategy of the kernel functions is constructed for estimating the kernel function incidence matrix, a shared representation matrix of the adverse reactions between drugs of a certain kernel function can be reconstructed by a shared representation matrix of the adverse reactions between drugs of other kernel functions, and a private representation matrix of the adverse reactions between drugs of a certain kernel function can also be reconstructed by a private representation matrix of the adverse reactions between drugs of other kernel functions, so that the reconstruction strategy of the kernel functions is obtained;
S4: constructing a multi-kernel representation learning model, selecting representative kernel functions, and constructing an optimal kernel function combination, specifically including: constructing an objective function of the multi-kernel representation learning model according to the distance learning strategy of the kernel functions and the reconstruction strategy of the kernel functions, solving the objective function to obtain the kernel function incidence matrix, so as to obtain a weight of each kernel function, sequencing the kernel functions according to the weights, selecting the kernel functions with the maximum weights as the representative kernel functions to further obtain a representative kernel function set and derive the optimal kernel function combination, and finally obtaining the multi-kernel representations of the shared and private representations of the drugs based on the optimal kernel function combination;
S5: constructing a model for predicting adverse reactions between drugs by using the optimal kernel function combination, specifically including: based on a vector r^{ij}, mining a potential relationship between the multi-attribute and multi-kernel representations of the drugs and the adverse reactions by using an R-layer neural network to obtain a mapping relationship between the multi-attribute and multi-kernel representations of the drugs and the adverse reactions between drugs, using concatenated multi-kernel representations of the shared and private representations of drugs dᵢ and dⱼ as an input of the neural network, using an output vector of the R^{th} layer of the neural network as a predicted value r̅^{ij} of the adverse reactions between drugs dᵢ and dⱼ, estimating an difference between the true value r^{ij} and the predicted value r̅^{ij} of adverse reaction vectors between drugs dᵢ and dⱼ by using a mean square error loss function, and performing training by using data collected in S1 to obtain a trained adverse reaction prediction model; and
S6: acquiring multi-attribute information of any two drugs, calculating the multi-kernel representations of the shared and private representations of two drugs, and inputting the multi-kernel representations into the trained adverse reaction prediction model to obtain a predicted result of the adverse reactions between drugs.

Further, the objective function constructed in S2 is as follows:

$$\begin{matrix}{min}_{P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] \\ s . t . P \geq 0 , {Q}^{m} \geq 0 , {U}^{m} \geq 0 , m = 1 , … , M\end{matrix}$$

where ∥⋅∥_{F}² denotes a Frobenius norm of a matrix, ∥⋅∥₀ denotes a norm of l₀ matrix (i.e., the number of non-0 elements in the matrix), a matrix P∈R^{N×E} denotes the shared representation of multiple attributes of the drugs, a matrix Q^{m}∈R^{N×E} denotes the private representation of the m^{th} attribute of the drugs, E denotes a dimension of the shared and private representations, U^{m}∈R^{E×L}^{ₘ} denotes a reconstructed coefficient matrix of an original feature space X^{m} of the shared and private representations for the m^{th} attribute, and α^{m} denotes a sparsity regularization parameter for the m^{th} attribute; and

the objective function can be formulated by augmented Lagrangian function and further solved by an alternating direction multipliers method and a non-negative matrix factorization optimization method so as to obtain iterative update solutions of the shared representation P and private representation Q^{m} of the multi-attribute feature space of the drugs and the reconstructed coefficient matrix U^{m} of the multi-attribute feature space, m=1, . . . , M, and the maximum number of iterations or a minimum change threshold of the objective function are set and variables are iteratively updated to obtain an optimal solution of the objective function.

Further, the distance learning strategy of the kernel functions constructed in S3 is as follows:

$$\begin{matrix}{min}_{Y} {\sum }_{l , s}^{L} ( {D}_{ls}^{P} + {D}_{ls}^{Q} ) {Y}_{ls} = tr [ ( {D}^{P} + {D}^{Q} ) Y ] \\ s . t . {Y}^{T} 1 = 1 , diag ( Y ) = 0 , Y \geq 0 ,\end{matrix}$$

where L denotes the number of kernel functions, Dₗₛ^{P} and Dₗₛ^{Q} denote the distances between the kernel functions κₗ and κₛ of the shared representation and the private representation, Yₗₛ is an element in the kernel function incidence matrix Y, and denotes a probability that kernel function κₗ can be used to represent kernel function κₛ, matrixes D^{P}, D^{Q}∈R^{L×L} respectively denote similarity matrixes of the L kernel functions in terms of the shared representation and the private representation, 1 and 0 respectively denote an all-1 vector and an all-0 vector, Y≥0 denotes non-negativity of all elements in the kernel function incidence matrix Y, and diag(Y)=0 denotes that diagonal elements of the matrix Y are 0;
the weight Wₗ of the kernel function κₗ is defined as

$${w}_{l} = \frac{1}{L} {\sum }_{s = 1}^{L} {y}_{ls} ;$$

and
the reconstruction strategy of kernel function is as follows:

$${min}_{Y} {\sum }_{s = 1}^{L} [ {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} ]$$

where κₗ^{P} is the shared representation matrix of the adverse reactions between drugs based on the kernel function κₗ, κₗ^{Q} is the private representation matrix of the adverse reactions, between drugs based on the kernel function κₗ, κₛ^{P} is the shared representation matrix of the adverse reactions between drugs based on the kernel function κₛ, and κₛ^{Q} is the private representation matrix of the adverse reactions between drugs based on the kernel function κₛ.

Further, the objective function of the multi-kernel representation learning model constructed in S4 is as follows:

$$\begin{matrix}{min}_{Y} {\sum }_{l = 1}^{L} [ {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} + λ tr [ ( {D}^{P} + {D}^{Q} ) Y ] \\ s . t . {Y}^{T} 1 = 1 , diag ( Y ) = 0 , Y \geq 0\end{matrix}$$

where the regularization parameter λ controls the weight of kernel function distance learning; the objective function of the multi-kernel representation learning model can be formulated by augmented Lagrangian function and is further optimized by an alternating direction multipliers method and a non-negative matrix factorization optimization method to obtain the iterative update solution of the kernel function incidence matrix Y, and by setting the maximum number of iterations or the minimum change threshold of the objective function, the matrix Y is iteratively updated and the optimal solution of the kernel function incidence matrix Y is obtained; and
the weight of the kernel function κₗ is obtained based on the optimal solution of Y and a definition of the weight Wₗ, the weights of the L kernel functions are sequenced, the r_{L} kernel functions with the maximum kernel function weights are selected as the representative kernel function set, and the multi-kernel representations κ_{w}(P_{i⋅}, P_{j⋅}) and κ_{w}(Q_{i⋅}, Q_{j⋅}) of the shared representation and the private representation of drugs dᵢ and dⱼ are obtained by using the representative kernel function set:

$${κ}_{w} ( {P}_{i \cdot } , {P}_{j \cdot } ) = {[ \begin{matrix}{w}_{1} {\sum }_{\overline{l} = 1}^{{r}_{L}} {Y}_{\overline{l} 1} {κ}_{\overline{l}} ( {P}_{i \cdot } , {P}_{j \cdot } ) , \\ {w}_{2} {\sum }_{\overline{l} = 1}^{{r}_{L}} {Y}_{\overline{l} 2} {κ}_{\overline{l}} ( {P}_{i \cdot } , {P}_{j \cdot } ) , \\ … \\ {w}_{L} {\sum }_{\overline{l} = 1}^{{r}_{L}} {Y}_{\overline{l} L} {κ}_{\overline{l}} ( {P}_{i \cdot } , {P}_{j \cdot } )\end{matrix} ]}^{T} , {κ}_{w} ( {Q}_{i \cdot } , {Q}_{j \cdot } ) = {[ \begin{matrix}{w}_{1} {\sum }_{\overline{l} = 1}^{{r}_{L}} {Y}_{\overline{l} 1} {κ}_{\overline{l}} ( {Q}_{i \cdot } , {Q}_{j \cdot } ) , \\ {w}_{2} {\sum }_{\overline{l} = 1}^{{r}_{L}} {Y}_{\overline{l} 2} {κ}_{\overline{l}} ( {Q}_{i \cdot } , {Q}_{j \cdot } ) , \\ … \\ {w}_{L} {\sum }_{\overline{l} = 1}^{{r}_{L}} {Y}_{\overline{l} L} {κ}_{\overline{l}} ( {Q}_{i \cdot } , {Q}_{j \cdot } )\end{matrix} ]}^{T}$$

where Y_{l̅l̅} denotes an element in the l̅^{th} row and the l^{th} column in the kernel function incidence matrix Y, l̅=1, 2, . . . , r_{L}.

Further, the mapping relationship between the multi-attribute and multi-kernel representations between drugs and the adverse reactions of the R-layer neural network in S5 is as follows:

$$\begin{matrix}{h}^{( 1 )} = σ ( {E}^{( 1 )} {κ}_{w}^{ij} + {b}^{( 1 )} ) , \\ {h}^{( r )} = σ ( {E}^{( r )} {h}^{( r - 1 )} + {b}^{( r )} ) , r = 2 , 3 , … , R\end{matrix}$$

where h^{(r)}, E^{(r)}, and b^{(r)} respectively denote an output vector, a coefficient matrix, and an offset vector of the r^{th} layer of the neural network, κ_{w}^{ij} denotes the concatenation of the multi-kernel representations of the shared private representations of drugs dᵢ and dⱼ, i.e., κ_{w}^{ij}=[κ_{w}(P_{i⋅}, P_{j⋅}), κ_{w}(Q_{i⋅}, Q_{j⋅})], the output vector h^{(R)} of the R^{th} layer of the neural network is the predicted value r^{ij} of the adverse reactions between drugs dᵢ and dⱼ, and the difference between the true value r^{ij} and the predicted value r̅^{ij} of the adverse reaction vectors between drugs dᵢ and dⱼ is estimated by using the mean square error loss function: and

$$L = {\sum }_{{‖ {r}^{ij} ‖}_{2} \geq 0} {‖ {r}^{ij} - {\overline{r}}^{ij} ‖}_{2}^{2}$$

the larger an element r̅ₖ^{ij} in the predicted value r̅^{ij} is, the higher the probability of the k^{th} adverse reaction induced by drugs dᵢ and dⱼ is.

The present invention has the following beneficial effects: compared with a conventional prediction method, in light of the differences of multiple attributes of the drugs in revealing the potential characteristics of the adverse reactions between drugs, the consistent optimal kernel function combination is constructed. The optimal kernel function embodies preference and tendencies of various kernel functions when calculating the multi-attribute similarities, and the potential relationship between the multi-attribute similarities of the drugs and the adverse reactions between drugs is established, thereby improving the accuracy of predicting the adverse reactions between drugs. The present invention is capable of providing data support to experimental research on the adverse reactions between drugs, improves the clinical experimental research of the adverse reactions between drugs, and is of great significance in promoting clinical medication safety.

### BRIEF DESCRIPTION OF THE DRAWINGS

FIG. 1 is an overall framework of the present invention.

FIG. 2 is an overall flowchart of the present invention.

FIGS. 3A-3C are schematic diagrams of partially predicted adverse reactions between drugs.

### DETAILED DESCRIPTION OF THE EMBODIMENTS

The present invention will be described in detail below in combination with drawings and specific embodiments.

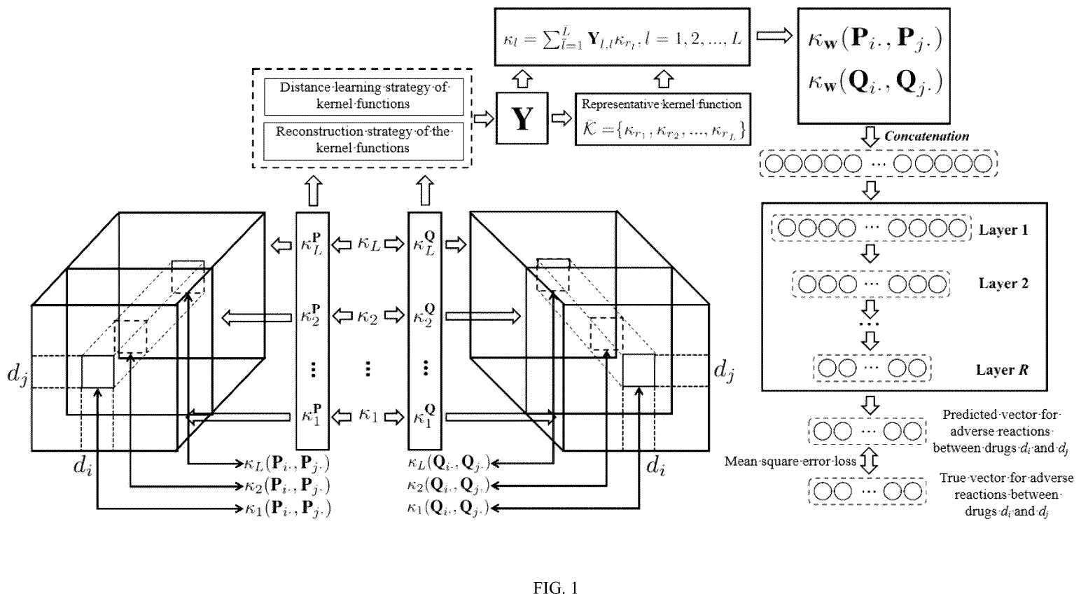

As shown in FIG. 1, in the present invention, prediction of adverse reactions between drugs is conducted based on multi-attribute and multi-kernel representation learning. The method includes: based on shared representation and private representation of multiple attributes of drugs, providing a multi-kernel representation learning model; designing a distance learning strategy of kernel functions and a reconstruction strategy of the kernel functions to select a representative kernel function; constructing an optimal kernel function combination by an incidence relationship among the kernel functions; and finally, predicting the adverse reactions between drugs based on multi-attribute and multi-kernel representations through a neural network. By constructing the optimal kernel function combination to reveal the potential characteristics of different attributes in modeling the adverse reactions between drugs, and integrating with preference and tendencies of various kernel functions when calculating the multi-attribute similarities, thereby improving the accuracy of predicting the adverse reactions between drugs. The present invention is capable of providing data support to experimental research on the adverse reactions between drugs, improves the clinical experimental research of the adverse reactions between drugs, and is of great significance in promoting clinical medication safety.

### Embodiment

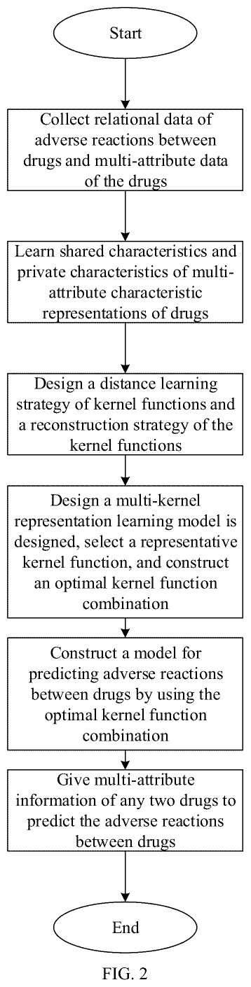

As shown in FIG. 2, the embodiment includes the following main steps:
S1: collecting data of adverse reactions between drugs and multi-attribute information of the drugs;
S2: learning shared and private representations of the multi-attribute information of drugs;
S3: constructing a distance learning strategy of kernel functions and a reconstruction strategy of the kernel functions;
S4: designing a multi-kernel representation learning model, selecting a representative kernel function; and constructing an optimal kernel function combination;
S5: constructing a model for predicting adverse reactions between drugs by using the optimal kernel function combination; and
S6: predicting the adverse reactions between drugs by multi-attribute information of any two drugs.

In the step S1, collecting the data of the adverse reactions between drugs and the multi-attribute information of the drugs. In the embodiment, data of the adverse reactions between drugs is collected from a TWOSIDES database [N. P. Tatonetti, P. P. Ye, D. Roxana, R. B. Altman, Data-driven prediction of drug effects and interactions, Science Translational Medicine 4 (125) (2012) 1-26]. The TWOSIDES data base records adverse reactions of the combined use of two drugs. In the embodiment, a relationship for predicting the adverse reactions between drugs is established by means of attribute data such as molecular structures, target, pathways, side effects, phenotypes, and diseases of the drugs. Molecular structure and target information of the drugs is originated from a DrugBank database [D. S. Wishart, Y. D. Feunang, A. C. Guo, et al. DrugBank 5.0: A major update to the DrugBank database for 2018[J]. Nucleic Acids Research, 2018, 46(D1): D1074-D1082.]. Pathway and disease information of the drugs is originated from a KEGG database [M. Kanehisa, M. Furumichi, Y. Sato, et al. KEGG: Integrating viruses and cellular organisms[J]. Nucleic Acids Research, 2021, 49(D1): D545-D551.]. Side effect information of the drugs is originated from an SIDER database [M. Kuhn, I. Letunic, L. J. Jensen, et al. The SIDER database of drugs and side effects[J]. Nucleic Acids Research, 2016, 44(D1): D1075-D1079.]. Phenotype information of the drugs is originated from a CTD database [A. P. Davis, C. J. Grondin, R. J. Johnson, et al. Comparative toxicogenomics database (CTD): Update 2021[J]. Nucleic Acids Research, 2021, 49(D1): D1138-D1143.]. Based on data of the adverse reactions between drugs and the multi-attribute data of the drugs, totally 1188258 kinds of adverse reactions between drugs are acquired in the embodiment, involving N=567 drugs and K=258 adverse reactions. Basically, common drugs and adverse reactions are covered. The data collected by using the collection method is of higher reliability.

A drug set D={d₁, d₂, . . . , d_{N}} is given, for adverse reactions between drugs dᵢ and dⱼ, constructing a vector r^{ij}∈{0, 1}^{K} to denote an adverse reaction relationship between the i^{th} aj, where K denotes the number of of the types of adverse reactions, drug dᵢ and the j^{th} drug dⱼ, where K denotes the number of of the types of adverse reactions, and if the k^{th} adverse reaction is induced by the interaction between dᵢ and dⱼ, then, rₖ^{ij}=1; otherwise, rₖ^{ij}=0. For the attribute data such as molecular structures, target, pathways, side effects, phenotypes, and diseases of the drugs, binary vectors are used to denote the attribute representation of the drugs. Vector elements 1 and 0 respectively denote whether the drugs contain representation information of corresponding attributes. The source databases and representation dimensions of the multi-attribute information of the drugs are shown in Table 1. A matrix X^{m}∈R^{N×L}^{ₘ} is used to denote a feature space of an m^{th} attribute, where Lₘ denotes the feature dimension of the m^{th} attribute, m=1,2, . . . , M and M denotes a number of attributes. By taking the feature space of the molecular structure of the drug as an example (m=1), molecular structure information of the drug is collected from the DrugBank data base to construct a feature space X¹∈R^{N×L}^{₁} of the molecular structure of the drug, with the feature dimension L₁=881 of the molecular structure. Therefore, the attributes of the molecular structure of the drug dᵢ can be denoted by 881-dimensional binary vectors. If the dᵢ contains a j^{th} sub-structure, Xᵢⱼ¹=1; and if the dᵢ does not contain the j^{th} sub-structure, Xᵢⱼ¹=0.

In the step S2 of the embodiment, the shared and private representation of multi-attribute representations of drugs are learned. As shown in Table 1, due to highly dimensional and sparse representations of different attributes of the drugs and differences in representations dimensions of different attributes, the multi-attribute feature spaces of the drugs are projected to a same low-dimensional dense space to learn the shared and private representations of the multi-attribute informative information of the drugs.

TABLE 1
Source database and representations dimension information
of the multi-attribute information of the drugs

| | Source | Characteristic |
|---|---|---|
| Drug attribute | database | dimension Lₘ |
| Molecular structure | DrugBank | 881 |
| Target | DrugBank | 497 |
| Pathway | KEGG | 396 |
| Side effect | SIDER | 3687 |
| Phenotype | CTD | 2193 |
| Disease | KEGG | 482 |

In the step, the shared representations denote that different attributes have consistent contribution information for predicting the adverse reactions between drugs, and the private representations denote that different attributes contain specific information of each attribute, which plays a supplementing role in predicting the adverse reactions between drugs. In the step, by establishing the potential relationship between the shared and private representations and the original feature space of the drug attributes, that is, the feature space of each attribute consists of the shared representations and respective private representations in the projected low-dimension sense space, consistency information and supplementary information of the multi-attribute feature space are revealed. Therefore, a objective function of the shared representations and private representations denoted by the multi-attribute representations of the drugs can be written as follows:

$$\begin{matrix}{min}_{P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] \\ s . t . P \geq 0 , {Q}^{m} \geq 0 , {U}^{m} \geq 0 , m = 1 , … , M\end{matrix} \qquad ( 1 )$$

∥⋅∥_{F}² denotes a Frobenius norm of a matrix, ∥⋅∥₀ denotes a norm of l₀ matrix (i.e., the number of non-0 elements in the matrix), a matrix P∈R^{N×E} denoted the shared representations of multiple attributes of the drugs, a matrix Q^{m}∈R^{N×E} denotes the private representations of the m^{th} attribute of the drugs, E denotes a dimension of the shared and private representations, U^{m}∈R^{E×L}^{ₘ} denotes a reconstructed coefficient matrix of an original feature space X^{m} of the shared and private representations for the m^{th} attribute. For the m^{th} attribute, the number of features contained in each drug is limited and is far less than the representations dimension Lₘ of the m^{th} attribute, so the feature space of the attribute is highly sparse. Therefore, an l₀-norm constraint of the coefficient matrix U^{m} is introduced in the equation (1) to control the sparsity of a reconstruction matrix (P+Q^{m})U^{m} of the original feature space based on the shared and private representations, where α^{m} denotes a sparsity regularization parameter. Constraint conditions P≥0, Q^{m}≥0, and U^{m}≥0 are used to maintain the non-negativity of the original feature space X^{m}. In the step, the feature spaces of the drug attributes are divided into the shared representations P and the private representations Q^{m}, the feature spaces of M attributes share the same shared representations P, and different feature spaces X^{m} have respective private representations Q^{m}. The shared representations and the private representations are reconstructed through a sparse coefficient matrix U^{m}.

The equation (1) can be formulated by the augmented Lagrangian function and further optimized by the alternating direction multipliers method (ADMM), and the non-negative matrix factorization optimization method so as to acquire iterative update solutions of the shared representations P and private representations Q^{m} of the multi-attribute feature space of the drug and the reconstructed coefficient matrix U^{m} of the multi-attribute feature space, m=1, . . . , M. The maximum number of iterations or a minimum change threshold of the objective function are set and variables are iteratively updated to obtain an optimal solution of the objective function.

In the step S3 of the embodiment, the distance learning strategy of kernel functions and the reconstruction strategy of the kernel functions are designed. Based on the shared representations P and the private representations Q^{m} of M attributes acquired in step 2, the shared representations of the drug dᵢ are denoted as P_{i⋅}, and the private representations of the m^{th} attribute of the drug dᵢ are denoted as Q_{i⋅}^{m}, m=1, . . . , M. The private representations of the M attribute spaces of the drug dᵢ are concatenated to acquire the private representation Q_{i⋅}=[Q_{i⋅}¹, Q_{i⋅}², . . . , Q_{i⋅}^{M}] of the drug dᵢ.

K={κ₁, κ₂, . . . , κ_{L}} is given to denote L kernel function sets, where κₗ denotes the l^{th} kernel function κₗ. First, the share and private representations of drugs are projected to the high-dimensional space with a projection function ϕₗ(⋅), and the inner products of two drug attribute representations are calculated in the high-dimensional space. For the adverse reactions between drugs dᵢ and dⱼ, the shared representations P_{i⋅}, P_{j⋅} are taken as inputs of the kernel function κₗ, to be regarded as similarity measures κₗ(P_{i⋅}, P_{j⋅})=ϕₗ(P_{i⋅})^{T}ϕₗ(P_{j⋅}), the private representations Q_{i⋅}, Q_{j⋅} of dᵢ and dⱼ are also taken as inputs of the kernel function κₗ, to be regarded as similarity measures κₗ(Q_{i⋅}, Q_{j⋅})=ϕₗ(Q_{i⋅})^{T}ϕₗ(Q_{j⋅}), l=1, 2, . . . , L. w=[w₁, w₂, . . . , w_{L}] is given to denote the weight vectors of L kernel functions. Therefore, multi-kernel representations of the shared representations and private representations of the drugs dᵢ and dⱼ can be written as follows:

$${κ}_{w} ( {P}_{i \cdot } , {P}_{j \cdot } ) = {[ \begin{matrix}{w}_{1} {κ}_{1} ( {P}_{i \cdot } , {P}_{j \cdot } ) , \\ {w}_{2} {κ}_{2} ( {P}_{i \cdot } , {P}_{j \cdot } ) , \\ … \\ {w}_{L} {κ}_{L} ( {P}_{i \cdot } , {P}_{j \cdot } )\end{matrix} ]}^{T} , {κ}_{w} ( {Q}_{i \cdot } , {Q}_{j \cdot } ) = {[ \begin{matrix}{w}_{1} {κ}_{1} ( {Q}_{i \cdot } , {Q}_{j \cdot } ) , \\ {w}_{2} {κ}_{2} ( {Q}_{i \cdot } , {Q}_{j \cdot } ) , \\ … \\ {w}_{L} {κ}_{L} ( {Q}_{i \cdot } , {Q}_{j \cdot } )\end{matrix} ]}^{T} \qquad ( 2 )$$

For the kernel function κₗ, κₗ^{P}, κₗ^{Q}∈R^{N×N} respectively denote similarity matrixes of the shared representations and the private representations of the adverse reactions between drugs based on the kernel function κₗ, with matrix elements [κₗ^{P}]ᵢⱼ=κₗ(P_{i⋅}, P_{j⋅}), [κₗ^{Q}]ᵢⱼ=κₗ(Q_{i⋅}, Q_{j⋅}).

To select the appropriate representative kernel function to construct the optimal kernel function combination, the distance between the kernel functions κₗ and κₛ can be regarded as the similarity between the kernel functions κₗ and κₛ. The less the distance between the kernel functions κₗ and κₛ is, the larger the similarity between the kernel functions κₗ and κₛ is, so the probability that the kernel function κₗ is capable of being used to represent the kernel function κₛ is higher. A kernel function incidence matrix Y is designed, and a matrix element Yₗₛ is a probability that the kernel function κₗ can be used to represent the kernel function κₛ. Therefore, for the similarity matrixes κₗ^{P} of the shared representations and κₗ^{Q} of the private representations in terms of the kernel function κₗ, the distance between the kernel functions κₗ and κₛ of the shared representations and the private representations can be denoted as follows:

$$\begin{matrix}{D}_{ls}^{P} = {‖ {κ}_{l}^{P} - {κ}_{s}^{P} ‖}_{F}^{2} \\ {D}_{ls}^{Q} = {‖ {κ}_{l}^{Q} - {κ}_{s}^{Q} ‖}_{F}^{2}\end{matrix} \qquad ( 3 )$$

The matrixes D^{P}, D^{Q}∈R^{L×L} respectively denote the similarity matrixes of the L kernel functions on the shared representations and the private representations. Therefore, in combination with the kernel function incidence matrix Y, the distance learning strategy of the kernel functions can be written in the following:

$$\begin{matrix}{min}_{Y} {\sum }_{l , s}^{L} ( {D}_{ls}^{P} + {D}_{ls}^{Q} ) {Y}_{ls} = tr [ ( {D}^{P} + {D}^{Q} ) Y ] \\ s . t . {Y}^{T} 1 = 1 , diag ( Y ) = 0 , Y \geq 0 ,\end{matrix} \qquad ( 4 )$$

1 and 0 respectively denote an all-1 vector and an all-0 vector, Y≥0 denotes non-negativity of elements in the kernel function incidence matrix Y, diag(Y)=0 denotes that the diagonal element of the matrix Y is 0,

$${\sum }_{l = 1}^{L} {Y}_{ls} = 1$$

guarantees that a probability sum of L kernel functions as the representative kernel functions to represent the kernel function κₛ is 1, equivalent to a bound term Y^{T}=1 in the equation (4). Yₗₛ is the probability that the kernel function κₗ represent the kernel function κₛ, the weight wₗ of the kernel function κₗ can be regarded as a mean value of the probability that κₗ serves as the representative kernel function to represent the L kernel functions, that is,

$${w}_{l} = \frac{1}{L} {\sum }_{s = 1}^{L} {Y}_{ls} .$$

To estimate the kernel function incidence matrix Y, the shared representations matrix κₗ^{P} of the adverse reactions between drugs based on the kernel function κₗ can be represented by the shared representations matrix κₛ^{P} of the adverse reactions between drugs of other kernel functions κₛ, and the private representations matrix κₗ^{Q} of the adverse reactions between drugs based on the kernel function κₗ can also be represented by the private representations matrix κₛ^{Q} of the adverse reactions between drugs of other kernel functions κₛ, s=1, 2, . . . , L, s≠l. Therefore, the reconstruction strategy of the kernel functions can be written as the following rule items:

$${min}_{Y} {\sum }_{l = 1}^{L} [ {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} ] \qquad ( 5 )$$

In the step S4 of the embodiment, the multi-kernel representation learning model is designed, the representative kernel functions are selected, and the optimal kernel function combination is constructed. Based on the distance learning strategy of the kernel functions given in the equation (4) and the reconstruction strategy of the kernel functions given in the equation (5), the multi-kernel representation learning model is constructed, and the objective function of the model can be written as follows:

$$\begin{matrix}{min}_{Y} {\sum }_{l = 1}^{L} {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} + λ tr [ ( {D}^{P} + {D}^{Q} ) Y ] \\ s . t . {Y}^{T} 1 = 1 , diag ( Y ) = 0 , Y \geq 0\end{matrix} \qquad ( 6 )$$

The regularization parameter λ controls the wight of the distance learning of the kernel functions. The objective function can be formulated by the augmented Lagrangian function and further optimized by the ADMM method, to acquire the iterative update solution of the kernel function incidence matrix Y. By setting a maximum number of iterations or a minimum change threshold of the objective function, matrix Y is iteratively updated to finally acquire the optimal solution of the objective function.

Based on the optimized kernel function incidence matrix Y, the weight wₗ of the kernel function κₗ can be denoted as

$${w}_{l} = \frac{1}{L} {\sum }_{s = 1}^{L} {Y}_{ls} .$$

The weights of the L kernel functions are sequenced, r_{L} kernel functions with the maximum weights of the kernel functions are selected as the representative kernel functions, and the representative kernel function set can be denoted as K̅={κᵣ_{₁}, κᵣ_{₂}, . . . , κᵣ_{_{L}}}. The similarity matrix of the shared representations of the adverse reactions between drugs of kernel function κₗ is reconstructed by the similarity matrix of the shared representations of the adverse reactions between drugs of the representative kernel functions, i.e.,

$${κ}_{l}^{P} = {w}_{l} {\sum }_{\overline{l} = 1}^{{r}_{L}} {Y}_{\overline{u}} {κ}_{\overline{l}}^{P} ,$$

and the private representations of the adverse reactions between drugs of kernel function κₗ can be also reconstructed by the similarity matrix of the private representations of the adverse reactions between drugs of the representative kernel functions, i.e.,

$${κ}_{l}^{Q} = {w}_{l} {\sum }_{\overline{l} = 1}^{{r}_{L}} {Y}_{\overline{u}} {κ}_{\overline{l}}^{Q} ,$$

where Y_{l̅l̅} denotes elements in the l̅^{th} row and l^{th} column in the kernel function incidence matrix Y,

$${κ}_{l}^{P} = {w}_{l} {\sum }_{\overline{l} = 1}^{{r}_{L}} {Y}_{\overline{u}} {κ}_{\overline{l}}^{P}$$

denotes the reconstruction of the similarity matrix κₗ^{P} of the shared representations of the adverse reactions between drugs of the kernel function κₗ by the similarity matrix κ_{l̅}^{P} of the shared representations of the adverse reactions between drugs of the representative kernel function κ_{l̅} and the element Y_{l̅l̅} denoting the probability of the representative kernel function κ_{l̅} representing kernel function κₗ, l̅=1, 2, . . . , r_{L} Therefore, based on the selected representative kernel functions, multi-kernel representations of the shared representations and the private representations of the drugs dᵢ and dⱼ can be written as follows:

$${κ}_{w} ( {P}_{i \cdot } , {P}_{j \cdot } ) = {[ \begin{matrix}{w}_{1} {\sum }_{\overline{l} = 1}^{{r}_{L}} {Y}_{\overline{l} 1} {κ}_{\overline{l}} ( {P}_{i \cdot } , {P}_{j \cdot } ) , \\ {w}_{2} {\sum }_{\overline{l} = 1}^{{r}_{L}} {Y}_{\overline{l} 1} {κ}_{\overline{l}} ( {P}_{i \cdot } , {P}_{j \cdot } ) , \\ … \\ {w}_{L} {\sum }_{\overline{l} = 1}^{{r}_{L}} {Y}_{\overline{l} L} {κ}_{\overline{l}} ( {P}_{i \cdot } , {P}_{j \cdot } )\end{matrix} ]}^{T} , \qquad ( 7 )$$

$${κ}_{w} ( {Q}_{i \cdot } , {Q}_{j \cdot } ) = {[ \begin{matrix}{w}_{1} {\sum }_{\overline{l} = 1}^{{r}_{L}} {Y}_{\overline{l} 1} {κ}_{\overline{l}} ( {Q}_{i \cdot } , {Q}_{j \cdot } ) , \\ {w}_{2} {\sum }_{\overline{l} = 1}^{{r}_{L}} {Y}_{\overline{l} 1} {κ}_{\overline{l}} ( {Q}_{i \cdot } , {Q}_{j \cdot } ) , \\ … \\ {w}_{L} {\sum }_{\overline{l} = 1}^{{r}_{L}} {Y}_{\overline{l} L} {κ}_{\overline{l}} ( {Q}_{i \cdot } , {Q}_{j \cdot } )\end{matrix} ]}^{T}$$

The representative kernel function set K̅={κᵣ_{₁}, κᵣ_{₂}, . . . , κᵣ_{_{L}}} is regarded as the optimal kernel function combination.

In the step S5 of the embodiment, the model for predicting adverse reactions between drugs is constructed by using the optimal kernel function combination. A vector r^{ij} is given to denote the adverse reaction relationship between the drugs dᵢ and dⱼ, and an R-layer neural network is designed to mine a potential relationship between the multi-attribute and multi-kernel representations of the drugs and the adverse reactions. The equation (7) gives the multi-kernel representations κ_{w}(P_{i⋅}, P_{j⋅}), κ_{w}(Q_{i⋅}, Q_{j⋅}) of the shared representations and the private representations of the drugs dᵢ and dⱼ based on the representative kernel functions, so that the mapping relationship between the multi-attribute and multi-kernel representations between drugs and the adverse reactions can be written as follows:

$$\begin{matrix}{h}^{( 1 )} = σ ( {E}^{( 1 )} {κ}_{w}^{ij} + {b}^{( 1 )} ) , \\ {h}^{( r )} = σ ( {E}^{( r )} {h}^{( r - 1 )} + {b}^{( r )} ) , r = 2 , 3 , … , R\end{matrix} \qquad ( 8 )$$

h^{(r)}, E^{(r)}, and b^{(r)} respectively denote the output vector, the coefficient matrix, and the offset vector of the r^{th} layer of the neural network. κ_{w}^{ij} denotes the concatenation of the multi-kernel representations of the shared representations and the private representations of the drugs dᵢ, and dⱼ, that is, κ_{w}^{ij}=[κ_{w}(P_{i⋅}, P_{j⋅}), κ_{w}(Q_{i⋅}, Q_{j⋅})]. Particularly, the output vector h^{(R)} of the R^{th} layer of the neural network can be regarded as the predicted value r̅^{ij} of the adverse reactions between the drugs dᵢ and dⱼ. The difference between the true value r^{ij} and the predicted value r̅^{ij} of the adverse reaction vectors between the drugs dᵢ and dⱼ are estimated by the mean square error loss function and the error function can be written as follows:

$$L = {\sum }_{{‖ {r}^{ij} ‖}_{2} \geq 0} {‖ {r}^{ij} - {\overline{r}}^{ij} ‖}_{2}^{2} \qquad ( 9 )$$

In the step S6 of the embodiment, multi-attribute information X_{a⋅}^{m} and X_{b⋅}^{m}, m=1, 2, . . . , M, of the drugs dₐ and d_{b} is given to predict the adverse reactions between the drugs dₐ and a_{b}. Through the step S2 of the embodiment, the shared representations P_{a⋅} and P_{b⋅} and the private representations Q_{a⋅}^{m}, Q_{b⋅}^{m}, of the multi-attribute information of the drugs dₐ and d_{b} are obtained, m=1, 2, . . . , M, and the private representations of M attributes of dₐ and d_{b} are concatenated, i.e. Q_{a⋅}=[Q_{a⋅}¹, Q_{a⋅}², . . . , Q_{a⋅}^{M}], Q_{b⋅}=[Q_{b⋅}¹, Q_{b⋅}², . . . , Q_{b⋅}^{M}]. Based on the kernel function set K={κ₁, κ₂, . . . , κ_{L}}, the similarity measures κₗ(P_{a⋅}, P_{b⋅}), κₗ(Q_{a⋅}, Q_{b⋅}), of the shared representations and the private representations of the drugs dₐ and a_{b} are calculated, l=1, 2, . . . , L. Then, based on the representative kernel function set K̅={κᵣ_{₁}, κᵣ_{₂}, . . . , κᵣ_{_{L}}} and the kernel function incidence matrix Y obtained in the step S5 of the embodiment, the multi-kernel representations κ_{w}(P_{a⋅}, P_{b⋅}) and κ_{w}(Q_{a⋅}, Q_{b⋅}) of the shared representations and the private representations of the drugs dₐ and d_{b} are calculated by the equation (7) to obtain the concatenated vector κ_{w}^{ab}=[κ_{w}(P_{a⋅}, P_{b⋅}), κ_{w}(Q_{a⋅}, Q_{b⋅})]. Finally, by taking the vector κ_{w}^{ab} as the input of the R^{th} layer neural network constructed in the step S5 of the embodiment, a predicted vector r̅^{ab} of the adverse reactions between the drugs dₐ and d_{b} is calculated. The larger the vector element r̅ₖ^{ab} is, the more probable the k^{th} adverse reaction between the drugs dₐ and d_{b} is caused.

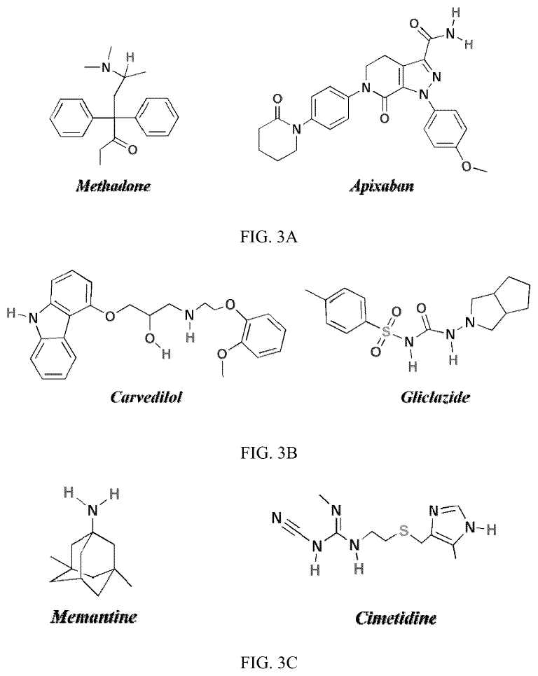

The method for predicting adverse reactions between drugs based on multi-attribute and multi-kernel representation learning provided in the embodiment, by constructing the optimal kernel function combination, explores the potential characteristic rules of different attributes in modeling the adverse reactions between drugs, and reveals the relationship between the multi-attribute similarities of the drugs and the adverse reactions between drugs, thereby realizing prediction of the adverse reactions between drugs. The predicted result can provide data support for research on the adverse reactions between drugs based on the bio-experimental method and research on the safety of the new drug. FIGS. 3A-3C show partially predicted adverse reactions between drugs, where FIG. 3A shows that combined use of a strong synthesized analgesic Methadone and an anticoagulant Apixaban will cause adverse reactions such as gingival bleeding, abnormal liver function, and malnutrition; FIG. 3B shows that simultaneous use of a hypotensive drug Carvedilol and an antidiabetic Gliclazide will induce adverse reactions such as pathoglycemia, ankylosis, uroclepsia, and angina; combined use of an opioid analgesic tramadol and an antifungal infection drug fluconazole will induce adverse reactions such as skin ulcer, hepatitis, and intestinal obstruction; and FIG. 3C shows that simultaneous use of a N-methyl-D-aspartic acid receptor antagonist Memantine for Alzheimer disease and a histamine type II receptor antagonist Cimetidine will cause adverse reactions such as psychiatric disorders nervous system tension.

What is claimed is:

1. A method for predicting adverse reactions between drugs based on a multi-attribute and multi-kernel representation learning, comprising the following steps:
S1: collecting data of the adverse reactions between the drugs and a multi-attribute information of the drugs to construct vectors of the adverse reactions between the drugs and the multi-attribute information of the drugs, comprising: defining a drug set as D={d₁, d₂, . . . , d_{N}}, wherein N is a number of the drugs; constructing a vector r^{ij}∈{0, 1}^{K} to denote an adverse reaction relationship between an i^{th} drug dᵢ and a j^{th} drug dⱼ, wherein K denotes a number of types of the adverse reactions, and if a k^{th} adverse reaction is induced by an interaction between the i^{th} drug dᵢ and the j^{th} drug dⱼ, then, rₖ^{ij}=1; otherwise, rₖ^{ij}=0, k=1, 2, . . . , K; and constructing a matrix X^{m}∈R^{N×L}^{ₘ} to denote a feature space of an m^{th} attribute of the drugs, wherein Lₘ denotes a feature dimension of the m^{th} attribute, m=1, 2, . . . , M and M denotes a number of attributes;
S2: learning a shared representation and a private representation of the multi-attribute information of the drugs, wherein the shared representation means that different attributes have a consistency information for a prediction of the adverse reactions between the drugs, the private representation means that the different attributes contain a specific supplementary information of each attribute, a feature space of the each attribute consists of the shared representation and the private representation after multi-attribute feature spaces of the drugs are projected to a same low-dimensional dense space, and an objective function is constructed based on a multi-attribute representation learning so as to obtain solutions of the shared representation and the private representation of M attribute spaces of the drugs;
S3: constructing a distance learning strategy of kernel functions and a reconstruction strategy of the kernel functions, comprising: using the shared representation and the private representation of the drugs as an input of a kernel function set, performing a similarity measure on the shared representation and the private representation between the drugs by the kernel function set to calculate distances among the kernel functions, setting a similarity to be increased as a decrease of the distances among the kernel functions, thus obtaining a similarity matrix of the shared representation and the private representation using the distances among the kernel functions of the shared representation and the private representation of the drugs, and finally, constructing a kernel function learning strategy according to the similarity matrix and a kernel function incidence matrix, wherein the kernel function incidence matrix is a probability matrix, and matrix entries denote a probability of a first kernel function representing a second kernel function; and the reconstruction strategy of the kernel functions is constructed for estimating the kernel function incidence matrix, a shared representation matrix of the adverse reactions between the drugs of a predetermined kernel function is configured to reconstructed by a shared representation matrix of the adverse reactions between the drugs of other kernel functions, and a private representation matrix of the adverse reactions between the drugs of the predetermined kernel function is configured to reconstructed by a private representation matrix of the adverse reactions between the drugs of the other kernel functions, so that the reconstruction strategy of the kernel functions is obtained;
S4: constructing a multi-kernel representation learning model, selecting a representative kernel function, and constructing an optimal kernel function combination, comprising: constructing an objective function of the multi-kernel representation learning model according to the distance learning strategy of the kernel functions and the reconstruction strategy of the kernel functions, solving the objective function of the multi-kernel representation learning model to obtain the kernel function incidence matrix, so as to obtain a weight of each kernel function, sequencing the kernel functions according to the weight of each kernel function, selecting a kernel function with a maximum weight as the representative kernel function to further obtain a representative kernel function set as the optimal kernel function combination, and finally obtaining multi-kernel representations of the shared representation and the private representation of the drugs based on the optimal kernel function combination;
S5: constructing a model for predicting the adverse reactions between the drugs by using the optimal kernel function combination, comprising: based on the vector r^{ij}, mining a potential relationship between multi-attribute and multi-kernel representations of the drugs and the adverse reactions by using an R-layer neural network to obtain a mapping relationship between the multi-attribute and multi-kernel representations of the drugs and the adverse reactions between the drugs, using concatenated multi-kernel representations of the shared representation and the private representation of the i^{th} drug dᵢ and the j^{th} drug dⱼ as an input of the R-layer neural network, using an output vector of an R^{th} layer of the R-layer neural network as a predicted value r̅^{ij} of the adverse reactions between the i^{th} drug dᵢ and the j^{th} drug dⱼ, estimating a difference between a true value r^{ij} and the predicted value r̅^{ij} of adverse reaction vectors between the i^{th} drug dᵢ and the j^{th} drug dⱼ by using a mean square error loss function, and performing a training by using the data collected in the S1 to obtain a trained adverse reaction prediction model; and
S6: acquiring the multi-attribute information of two drugs, calculating the multi-kernel representations of the shared representation and the private representation of the two drugs, and inputting the multi-kernel representations into the trained adverse reaction prediction model to obtain a predicted result of the adverse reactions between the two drugs.

2. The method for predicting the adverse reactions between the drugs based on the multi-attribute and multi-kernel representation learning according to claim 1, wherein the objective function constructed in the S2 is as follows:
$$\begin{matrix}{min}_{P , {Q}^{m} , {U}^{m}} {\sum }_{m = 1}^{M} [ {‖ {X}^{m} - ( P + {Q}^{m} ) {U}^{m} ‖}_{F}^{2} + {α}^{m} {‖ {U}^{m} ‖}_{0} ] \\ s . t . P \geq 0 , {Q}^{m} \geq 0 , {U}^{m} \geq 0 , m = 1 , … , M\end{matrix}$$
wherein ∥⋅∥_{F}² denotes a Frobenius norm of a matrix, ∥⋅∥₀ denotes an l₀ norm of the matrix (i.e., a number of non-0 elements in the matrix), a matrix P∈R^{N×E} denotes a shared representation of multiple attributes of the drugs, a matrix Q^{m}∈R^{N×E} denotes a private representation of the m^{th} attribute of the drugs, E denotes a dimension of the shared representation and the private representation, U^{m}∈R^{E×L}^{ₘ} denotes a reconstructed coefficient matrix of an original feature space X^{m} of the shared representation and the private representation for the m^{th} attribute, and α^{m} denotes a sparsity regularization parameter for the m^{th} attribute; and
the objective function can be formulated by an augmented Lagrangian function and further solved by an alternating direction multipliers method and a non-negative matrix factorization optimization method so as to obtain iterative update solutions of a shared representation P and a private representation Q^{m} of the multi-attribute feature spaces of the drugs and a reconstructed coefficient matrix U^{m} of the multi-attribute feature spaces, m=1, . . . , M, and a maximum number of iterations or a minimum change threshold of the objective function are set and variables are iteratively updated to obtain an optimal solution of the objective function.

3. The method for predicting the adverse reactions between the drugs based on the multi-attribute and multi-kernel representation learning according to claim 2, wherein the distance learning strategy of the kernel functions constructed in the S3 is as follows:
$$\begin{matrix}{min}_{Y} {\sum }_{l , s}^{L} ( {D}_{ls}^{P} + {D}_{ls}^{Q} ) {Y}_{ls} = tr [ ( {D}^{P} + {D}^{Q} ) Y ] \\ s . t . {Y}^{T} 1 = 1 , diag ( Y ) = 0 , Y \geq 0 ,\end{matrix}$$
wherein L denotes a number of the kernel functions, Dₗₛ^{P} and Dₗₛ^{Q} denote distances between the a kernel function κₗ and a kernel function κₛ of the shared representation and the private representation, Yₗₛ is an element in a kernel function incidence matrix Y, and denotes a probability of representing the kernel function κₛ by the kernel function κₗ, matrixes D^{P}, D^{Q}∈R^{L×L} respectively denote similarity matrixes of L kernel functions in terms of the shared representation and the private representation, 1 and 0 respectively denote an all-1 vector and an all-0 vector, Y≥0 denotes non-negativity of all elements in the kernel function incidence matrix Y, and diag(Y)=0 denotes that diagonal elements of the kernel function incidence matrix Y are 0;
a weight wₗ of the kernel function κₗ is defined as
$${w}_{l} = \frac{1}{L} {\sum }_{s = 1}^{L} {Y}_{ls} ;$$
and the reconstruction strategy of the kernel functions is as follows:
$${min}_{Y} {\sum }_{l , s}^{L} [ {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} ]$$
wherein κₗ^{P} is the shared representation matrix of the adverse reactions between the drugs based on the kernel function κₗ, κₗ^{Q} is the private representation matrix of the adverse reactions between the drugs based on the kernel function κₗ, κₛ^{P} is the shared representation matrix of the adverse reactions between the drugs based on the kernel function κₛ, and κₛ^{Q} is the private representation matrix of the adverse reactions between the drugs based on the kernel function κₛ.

4. The method for predicting the adverse reactions between the drugs based on the multi-attribute and multi-kernel representation learning according to claim 3, wherein the objective function of the multi-kernel representation learning model constructed in the S4 is as follows:
$$\begin{matrix}{min}_{Y} {\sum }_{l = 1}^{L} {‖ {κ}_{l}^{P} - {\sum }_{s = 1}^{L} {Y}_{sl} {κ}_{s}^{P} ‖}_{F}^{2} + {‖ {κ}_{l}^{Q} - {\sum }_{s = 1}^{L} {Y}_{sl} {κ}_{s}^{Q} ‖}_{F}^{2} + λ tr [ ( {D}^{P} + {D}^{Q} ) Y ] \\ s . t . {Y}^{T} 1 = 1 , diag ( Y ) = 0 , Y \geq 0\end{matrix}$$
wherein a regularization parameter λ controls a weight of a kernel function distance learning; the objective function of the multi-kernel representation learning model is formulated by the augmented Lagrangian function and is further optimized by the alternating direction multipliers method and the non-negative matrix factorization optimization method to obtain an iterative update solution of the kernel function incidence matrix Y, and by setting a maximum number of iterations or a minimum change threshold of the objective function of the multi-kernel representation learning model, the kernel function incidence matrix Y is iteratively updated and an optimal solution of the kernel function incidence matrix Y is obtained; and
a weight of the kernel function κₗ is obtained based on the optimal solution of the kernel function incidence matrix Y and a definition of the weight wₗ, weights of the L kernel functions are sequenced, r_{L} kernel functions with maximum kernel function weights are selected as the representative kernel function set, and multi-kernel representations κ_{w}(P_{i⋅}, P_{j⋅}) and κ_{w}(Q_{i⋅}, Q_{j⋅}) of the shared representation and the private representation of the i^{th} drug dᵢ and the j^{th} drug dⱼ are obtained by using the representative kernel function set:
$${κ}_{w} ( {P}_{i \cdot } , {P}_{j \cdot } ) = {[ \begin{matrix}{w}_{1} {\sum }_{\overline{l} = 1}^{{r}_{L}} {Y}_{\overline{l} 1} {κ}_{\overline{l}} ( {P}_{i \cdot } , {P}_{j \cdot } ) , \\ {w}_{2} {\sum }_{\overline{l} = 1}^{{r}_{L}} {Y}_{\overline{l} 2} {κ}_{\overline{l}} ( {P}_{i \cdot } , {P}_{j \cdot } ) , \\ … \\ {w}_{L} {\sum }_{\overline{l} = 1}^{{r}_{L}} {Y}_{\overline{l} L} {κ}_{\overline{l}} ( {P}_{i \cdot } , {P}_{j \cdot } )\end{matrix} ]}^{T} , \qquad ( 7 )$$
$${κ}_{w} ( {Q}_{i \cdot } , {Q}_{j \cdot } ) = {[ \begin{matrix}{w}_{1} {\sum }_{\overline{l} = 1}^{{r}_{L}} {Y}_{\overline{l} 1} {κ}_{\overline{l}} ( {Q}_{i \cdot } , {Q}_{j \cdot } ) , \\ {w}_{2} {\sum }_{\overline{l} = 1}^{{r}_{L}} {Y}_{\overline{l} 2} {κ}_{\overline{l}} ( {Q}_{i \cdot } , {Q}_{j \cdot } ) , \\ … \\ {w}_{L} {\sum }_{\overline{l} = 1}^{{r}_{L}} {Y}_{\overline{l} L} {κ}_{\overline{l}} ( {Q}_{i \cdot } , {Q}_{j \cdot } )\end{matrix} ]}^{T}$$
wherein Y_{l̅l̅} denotes an element in an l̅^{th} row and an l^{th} column in the kernel function incidence matrix Y, l̅=1, 2, . . . , r_{L}.

5. The method for predicting the adverse reactions between the drugs based on the multi-attribute and multi-kernel representation learning according to claim 4, wherein the mapping relationship between the multi-attribute and multi-kernel representations of the drugs and the adverse reactions of the R-layer neural network in the S5 is as follows:
$$\begin{matrix}{h}^{( 1 )} = σ ( {E}^{( 1 )} {κ}_{w}^{ij} + {b}^{( 1 )} ) , \\ {h}^{( r )} = σ ( {E}^{( r )} {h}^{( r - 1 )} + {b}^{( r )} ) , r = 2 , 3 , … , R\end{matrix}$$
wherein h^{(r)}, E^{(r)}, and b^{(r)} respectively denote the output vector, a coefficient matrix, and an offset vector of the R^{th} layer of the R-layer neural network, κ_{w}^{ij} denotes a concatenation of the multi-kernel representations of the shared representation and the private representation of the i^{th} drug dᵢ and j^{th} drug dⱼ, i.e., κ_{w}^{ij}=[κ_{w}(P_{i⋅}, P_{j⋅}), κ_{w}(Q_{i⋅}, Q_{j⋅})], the output vector h^{(R)} of the R^{th} layer of the R-layer neural network is the predicted value r̅^{ij} of the adverse reactions between the i^{th} drug dᵢ and the j^{th} drug dⱼ, and the difference between the true value r^{ij} and the predicted value r̅^{ij} of the adverse reaction vectors between the i^{th} drug dᵢ and the j^{th} drug dⱼ is estimated by using the mean square error loss function:
$$L = {\sum }_{{‖ {r}^{ij} ‖}_{2} \geq 0} {‖ {r}^{ij} - {\overline{r}}^{ij} ‖}_{2}^{2}$$
the larger an element r̅ₖ^{ij} in the predicted value r̅^{ij} is, the higher a probability of the k^{th} adverse reaction induced by the i^{th} drug dᵢ and the j^{th} drug dⱼ is.
